(19) United States
(12) Patent Application Publication
(10) Pub. No.: US 2011/0189274 A1
(43) Pub. Date: Aug. 4, 2011

(54) Stable Pharmaceutical Compositions Of Montelukast Or Its Salts Or Solvates Or Hydrates

(76) Inventors: Swati Mukherjee, Haryana (IN); Gour Mukherji, Haryana (IN)

(21) Appl. No.: 13/119,447

(22) PCT Filed: Oct. 6, 2009
(86) PCT No.: PCT/IN2009/000554

§ 371 Date: Mar. 17, 2011

(30) Foreign Application Priority Data
Oct. 6, 2008 (IN) 2314/DEL/2008

(51) Int. Cl. A61K 9/20; A61K 31/47; A61P 11/00

(52) U.S. Cl. 424/464; 514/311

(57) ABSTRACT

A stable pharmaceutical composition includes a therapeutically effective amount of montelukast or its pharmaceutically acceptable salts, solvates or hydrates and a blend which includes microcrystalline cellulose and a flavoring agent and wherein said composition is substantially free of montelukast sulfoxide degradation product. A process for preparing such composition is also disclosed.

### FIELD OF THE INVENTION

This invention, in general relates to a pharmaceutical composition comprising montelukast. In particular, the present invention provides a stable pharmaceutical composition comprising montelukast or its pharmaceutically acceptable salts or solvates or hydrates, wherein said composition is substantially free of montelukast sulfoxide degradation product.

### BACKGROUND OF THE INVENTION

Montelukast is a generic name for [R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropaneacetic acid. It is typically provided as monosodium salt, which is represented by the formula I

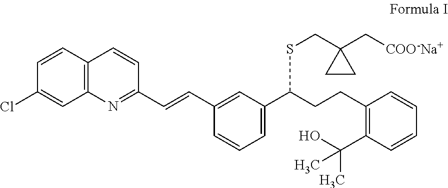

Montelukast sodium is a hygroscopic, optically active and white to off-white powder. Montelukast sodium is freely soluble in ethanol, methanol, and water and practically insoluble in acetonitrile.

Montelukast sodium is an orally active selective leukotriene receptor antagonist that inhibits the cysteinyl leukotriene CysLT₁ receptor. Leukotrienes are associated with the inflammation and constriction of airway muscles and the accumulation of fluid in the lungs. U.S. Pat. No. 5,565,473 to Belley et al. discloses a genus of pharmaceutically useful compounds, which belong to leukotriene antagonist and act as anti-asthmatic, anti-allergic, anti-inflammatory, and cyctoprotective agents. Example 161 in conjunction with example 146, of U.S. Pat. No. 5,565,473, discloses the synthesis of montelukast sodium.

Montelukast sodium is commercially marketed as Singulair® by Merck & Co., Inc. It is formulated as oral granules of strength 4 mg, chewable tablets of strength 4 mg and 5 mg and film coated tablets of strength 10 mg. Film coated Singulair tablets contain montelukast sodium along with inactive ingredients as microcrystalline cellulose, lactose monohydrate, croscarmellose sodium, hydroxypropylcellulose and magnesium stearate. The film coating consists of hydroxypropyl methylcellulose, hydroxypropylcellulose, titanium dioxide, red ferric oxide, yellow ferric oxide, and carnauba wax. The chewable Singulair tablets contain montelukast sodium along with inactive ingredients: mannitol, microcrystalline cellulose, hydroxypropylcellulose, red ferric oxide, croscarmellose sodium, cherry flavor, aspartame and magnesium stearate. The Singulair granules contain montelukast sodium along with inactive ingredients: mannitol, hydroxypropyl cellulose and magnesium stearate. Singulair is approved by USFDA for the prophylaxis and chronic treatment of Asthma in humans.

Pharmaceuticals intended for oral administration are typically provided in solid dosage form as tablets, capsules, pills, lozenges, or granules. Tablets are swallowed whole, chewed in the mouth, or dissolved in the oral cavity. Chewable or disintegrative tablets are often employed in the administration of pharmaceuticals where it is impractical to provide a tablet for swallowing whole, for instance with pediatric patients and geriatric patients. In addition, with chewable tablets, the act of chewing helps to break up the tablet particles as the tablet disintegrates and may increase the rate of absorption by the digestive tract. In addition, chewable tablets avoid mishaps that may occur with liquids, such as spillage and stains.

U.S. Pat. No. 5,489,436 discloses chewable tablet comprising a medicament coated with a taste masking effective amount of a polymer blend of at least 5% by weight of a blend of dimethylaminoethyl methacrylate and neutral methacrylic acid ester (MM/MAE) and a cellulose ester (CE). Preferably, the cellulose ester (CE) is at least one of cellulose acetate (CA), cellulose acetate butyrate (CAB) or cellulose triacetate (CTA).

U.S. Pat. No. 6,932,979 discloses chewable tablet comprising pharmaceutically active ingredient; chewable component selected from gum arabic, tragacanth, guar gum, dry glucose syrup, dry fructose syrup and carboxymethyl cellulose; one syrup component selected from carbohydrate syrup, concentrated maltodextrin solution and sugar alcohol syrup; and at least one fat or wax component.

U.S. Pat. No. 6,753,009 discloses chewable dosage form comprising pharmaceutically active ingredient and a matrix comprising polyethylene oxide.

U.S. Pat. No. 6,471,991 discloses compressed, chewable, convex shaped tablet comprising active ingredient; water-disintegratable, compressible carbohydrate; a binder and such tablets having a hardness of 2 to 11 kp/cm².

U.S. Pat. No. 5,225,197 discloses chewable tablet comprising medicament, chewable base selected from mannitol, sorbitol, dextrose, fructose and lactose; and an effervescent couple comprising an acidic component and a basic component.

U.S. Pat. No. 5,760,094 discloses chewable tablet comprising an ingredient requiring taste masking and hydrolyzed gelatin to mask the taste of said ingredient.

US20070184108 discloses stable pharmaceutical compositions comprising montelukast or a salt thereof and a pharmaceutically acceptable excipient selected from at least one of diluent, binder, disintegrant, lubricant and glidant, provided that the pharmaceutically acceptable excipient is not microcrystalline cellulose. The said application teaches that microcrystalline cellulose excipient is deleterious for the stability of the montelukast composition as it leads to the formation of sulfoxide impurity, owing to reaction between montelukast and microcrystalline cellulose.

However, microcrystalline cellulose is a favorable excipient for the preparation of immediate and sustained release pharmaceutical compositions, because of its versatile functionality and easy availability.

The pharmaceutical compositions suitable for oral administration to humans and containing montelukast sodium must have constant chemical and physical properties complying with demanding requirements and regulations of health and medicine regulatory agencies across the world, especially USFDA, MHRA and TGA.

There is an unmet need to produce stabilized pharmaceutical composition of montelukast sodium substantially free of montelukast sulfoxide degradation product.

### OBJECTS AND SUMMARY OF THE INVENTION

It is a principal object of the present invention to provide a stable pharmaceutical composition of montelukast or its pharmaceutically acceptable salts, solvates or hydrates wherein the composition comprises a therapeutically effective amount of montelukast or its pharmaceutically acceptable salts, solvates or hydrates and a blend comprising microcrystalline cellulose and a flavoring agent.

It is another object of the present invention to provide a stable pharmaceutical composition of montelukast or its pharmaceutically acceptable salts, solvates or hydrates wherein the composition comprises of a therapeutically effective amount of montelukast or its pharmaceutically acceptable salts, solvates or hydrates and a blend comprising microcrystalline cellulose and a flavoring agent, wherein said composition contains minimized amount of montelukast sulfoxide degradation product.

It is one other object of the present invention, wherein the invention provides a process for preparing such stable pharmaceutical composition comprising montelukast or its pharmaceutically acceptable salts, solvates or hydrates.

The above and other objects of the present invention are achieved by employing the following preferred embodiments, however the scope of the present invention will not be limited in accordance with the disclosed embodiments herein below.

In accordance with a preferred embodiment of the present invention, there is provided a stable pharmaceutical composition, wherein the stable pharmaceutical composition comprises a therapeutically effective amount of montelukast or its pharmaceutically acceptable salts, solvates or hydrates and a blend comprising microcrystalline cellulose and a flavoring agent, wherein said composition contains less than about 0.1% by weight of montelukast sulfoxide degradation product after storage at about 40° C. and about 75% relative humidity for three months. Preferably the composition contains less than about 0.04% by weight of montelukast sulfoxide degradation product.

In accordance with another embodiment of the present invention, there is provided a stable pharmaceutical composition, wherein said composition comprises a therapeutically effective amount of montelukast or its pharmaceutically acceptable salts, solvates or hydrates and a blend comprising microcrystalline cellulose and a flavoring agent, and wherein said composition contains less than about 0.1% by weight of montelukast sulfoxide degradation product after storage at about 40° C. and about 75% relative humidity for three months, wherein the flavoring agent is selected from strawberry, cherry, banana and other flavors or mixture thereof. Preferably the composition contains less than about 0.04% by weight of montelukast sulfoxide degradation product.

In accordance with one other embodiment of the present invention, there is provided a stable pharmaceutical composition, wherein said composition comprises a therapeutically effective amount of montelukast or its pharmaceutically acceptable salts, solvates, or hydrates and a blend comprising microcrystalline cellulose and a strawberry flavor, wherein said composition contains less than about 0.1% by weight of montelukast sulfoxide degradation product after storage at about 40° C. and about 75% relative humidity for three months. Preferably the composition contains less than about 0.04% by weight of montelukast sulfoxide degradation product.

In accordance with yet another embodiment of the present invention, there is provided a stable pharmaceutical composition, wherein said composition comprises a therapeutically effective amount of montelukast or its pharmaceutically acceptable salts, solvates or hydrates and a blend comprising microcrystalline cellulose and a flavoring agent, wherein the composition contains less than about 1% by weight of montelukast sulfoxide degradation product after storage at about 55° C. for 48 hours. Preferably the composition contains less than about 0.3% by weight of montelukast sulfoxide degradation product.

In accordance with yet another embodiment of the present invention, there is provided a stable pharmaceutical composition, wherein said composition comprises a therapeutically effective amount of montelukast or its pharmaceutically acceptable salts, solvates or hydrates and a blend comprising microcrystalline cellulose and a flavoring agent, wherein the composition contains less than about 1% by weight of montelukast sulfoxide degradation product after storage at about 55° C. for 48 hours, wherein the flavoring agent is selected from strawberry, cherry, banana and other flavors or mixture thereof. Preferably the composition contains less than about 0.3% by weight of montelukast sulfoxide degradation product.

In accordance with still another embodiment of the present invention, there is provided a stable pharmaceutical composition, wherein the montelukast is used in the form of montelukast sodium.

In accordance with still another embodiment of the present invention, there is provided a stable pharmaceutical composition, wherein the composition further comprising pharmaceutical acceptable excipients selected from diluents, binders, disintegrants, sweeteners, surfactants, lubricants, anti-tacking agents or colorants or mixture thereof.

In accordance with still another embodiment of the present invention, there is provided a stable pharmaceutical composition, wherein the composition further comprising mannitol, croscarmellose sodium, acesulfame potassium, red iron oxide, magnesium stearate or silicon dioxide or mixture thereof.

In accordance with still another embodiment of the present invention, there is provided a stable pharmaceutical composition, wherein said composition is formulated into solid dosage forms, preferably in coated, uncoated or chewable tablet forms. Chewable tablets are preferably, a multilayer tablet and more preferably a bilayer tablet.

In accordance with still another embodiment of the present invention, there is provided a stable pharmaceutical composition, wherein said composition is formulated into solid dosage forms, preferably a tablet, wherein the tablet is prepared by a process comprising dry granulation, wet granulation or direct compression.

In accordance with still another embodiment of the present invention, there is provided a stable pharmaceutical composition, wherein said composition is formulated into solid dosage forms, preferably a tablet, wherein the tablet is prepared by a process comprising sifting montelukast salt, solvates or hydrates microcrystalline cellulose, flavoring agent, diluent, disintegrant, binder, coloring agent, lubricant and glidants through a suitable size mesh; blending flavoring agent with microcrystalline cellulose in a blender and subsequently mixing the resultant blend with montelukast sodium, diluent, disintegrant, binder, and coloring agent; lubricating the resultant blend by mixing with lubricant and glidants; compressing the resultant blend obtained on a suitable tablet press.

In accordance with still another embodiment of the present invention, there is provided a stable pharmaceutical composition, wherein said composition is prepared by a process comprising providing montelukast salt, solvates or hydrates; blending flavoring agent with microcrystalline cellulose in a blender and subsequently mixing the resultant blend with montelukast sodium, solvates or hydrates; and preparing the composition enabling the stable pharmaceutical composition.

In accordance with still another embodiment of the present invention, there is provided a stable pharmaceutical composition, wherein said composition is prepared by a process comprising providing montelukast salt, solvates or hydrates; blending strawberry flavor with microcrystalline cellulose in a blender and subsequently mixing the resultant blend with montelukast sodium, solvates or hydrates; and preparing the composition enabling the stable pharmaceutical composition.

The stable pharmaceutical compositions achieved according to the present invention are useful for the treatment of the diseases such as asthma, allergic rhinitis and perennial allergic rhinitis.

### DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

As used herein, the term “stable” refers to significantly low or substantially free of montelukast sulfoxide degradation product in the pharmaceutical composition of the invention. Preferably, stable composition of the invention contains less than about 0.1%, more preferably less than about 0.04% of montelukast sulfoxide degradation product.

Montelukast sodium, used in preparing Singulair® tablets, is a hygroscopic material and easily absorbs up to 3 equivalents of water. The pharmaceutical compositions of montelukast available in US market are unstable in terms of presence of degradation product. The inventors of the present invention have surprisingly found that stable pharmaceutical compositions of montelukast can be prepared even in the presence of microcrystalline cellulose. Montelukast sodium compositions of the present invention are more stable than prior art compositions when stored for 3 months at about 40° C. and about 75% relative humidity (“accelerated storage conditions”) and when stored for 48 hours at about 55° C.

As used herein, the term “degradation product” refers to montelukast sulfoxide formed from decomposition of montelukast sodium during manufacturing and storage and frequently referred herein, montelukast sulfoxide degradation product.

As used herein, the term “montelukast sulfoxide degradation product” refers to [R-(E)]-1-[[[1-[3-[2-(7-chloro-2-quinolinyl)ethenyl]phenyl]-3-[2-(1-hydroxy-1-methylethyl)phenyl]propyl]sulfoxo]methyl]cyclopropane acetic acid represented by formula II

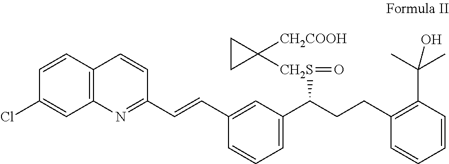

As used herein, the term “composition”, as in pharmaceutical composition, is intended to encompass a drug product comprising montelukast salts, solvates or hydrates, microcrystalline cellulose, specific flavoring agents and the other inert ingredient(s) (pharmaceutically acceptable excipients) that make up the carrier. Such pharmaceutical compositions are synonymous with “formulation” and “dosage form”. Pharmaceutical compositions of the invention include, but are not limited to, granules, tablets, capsules, powders and the like. Preferably, the pharmaceutical composition refers to tablets. More preferably, the pharmaceutical composition refers to chewable tablets in the single or bilayer form.

In main embodiment of the present invention, there is provided a stable pharmaceutical composition comprising a therapeutically effective amount of montelukast or its pharmaceutically acceptable salts, solvates or hydrates, wherein the composition comprises of microcrystalline cellulose and a specific flavoring agent. The invention also provides a process for preparing such a pharmaceutical composition.

According to one embodiment, the stable pharmaceutical composition comprises a therapeutically effective amount of montelukast or its pharmaceutically acceptable salts, solvates or hydrates and a blend comprising microcrystalline cellulose and a flavoring agent, wherein said composition contains less than about 0.1% by weight of montelukast sulfoxide degradation product after storage at about 40° C. and about 75% relative humidity for three months. Preferably the composition contains less than about 0.04% by weight of montelukast sulfoxide degradation product.

According to another embodiment, the stable pharmaceutical composition comprises a therapeutically effective amount of montelukast or its pharmaceutically acceptable salts, solvates or hydrates and a blend comprising microcrystalline cellulose and a flavoring agent, wherein said composition contains less than about 0.1% by weight of montelukast sulfoxide degradation product after storage at about 40° C. and about 75% relative humidity for three months, wherein the flavoring agent is selected from strawberry, cherry, banana and other flavors or mixture thereof. Preferably the composition contains less than about 0.04% by weight of montelukast sulfoxide degradation product.

According to another embodiment, the stable pharmaceutical composition comprises a therapeutically effective amount of montelukast or its pharmaceutically acceptable salts, solvates, or hydrates and a blend comprising microcrystalline cellulose and a strawberry flavor, wherein said composition contains less than about 0.1% by weight of montelukast sulfoxide degradation product after storage at about 40° C. and about 75% relative humidity for three months. Preferably the composition contains less than about 0.04% by weight of montelukast sulfoxide degradation product.

According to one other embodiment, the stable pharmaceutical composition comprises a therapeutically effective amount of montelukast or its pharmaceutically acceptable salts, solvates or hydrates and a blend comprising microcrystalline cellulose and flavoring agent, wherein the composition contains less than about 1% by weight of montelukast sulfoxide degradation product after storage at about 55° C. for 48 hours. Preferably the composition contains less than about 0.3% by weight of montelukast sulfoxide degradation product.

According to yet another embodiment, the stable pharmaceutical composition comprises a therapeutically effective amount of montelukast or its pharmaceutically acceptable salts, solvates or hydrates and a blend comprising microcrystalline cellulose and a flavoring agent, wherein the composition contains less than about 1% by weight of montelukast sulfoxide degradation product after storage at about 55° C. for 48 hours, wherein the flavoring agent is selected from strawberry, cherry, banana and other flavors or mixture thereof Preferably the composition contains less than about 0.3% by weight of montelukast sulfoxide degradation product.

According to still another embodiment, of the present invention, there is provided a stable pharmaceutical composition, wherein said composition is prepared by a process comprising:

providing montelukast salt, solvates or hydrates;

blending flavoring agent with microcrystalline cellulose in a blender and subsequently mixing the resultant blend with montelukast sodium, solvates or hydrates; and

preparing the composition enabling the stable pharmaceutical composition.

According to still another embodiment, of the present invention, there is provided the stable pharmaceutical composition, wherein said composition is prepared by a process comprising:

providing montelukast salt, solvates or hydrates;

blending strawberry flavor with microcrystalline cellulose in a blender and subsequently mixing the resultant blend with montelukast sodium, solvates or hydrates; and

preparing the composition enabling the stable pharmaceutical composition.

According to the invention, the degradation product, montelukast sulfoxide is expressed as weight percentage of the labeled (initial) amount of montelukast in the composition.

“Therapeutically effective amount” of montelukast as used herein refers to amount of montelukast or its pharmaceutically acceptable salts or solvates or hydrates necessary to elicit desired therapeutic response in the mammal. The usual dose of montelukast as approved by USFDA is 4 to 10 mg daily in the form of oral tablet, chewable tablet and oral granules. Actual dose of the drug is decided based on number of factors, including the patient's age, weight and severity of the disease.

The stable pharmaceutical compositions of the present invention preferably comprises sodium salt of montelukast.

The stable pharmaceutical compositions of the present invention are formulated into solid dosage forms, preferably in coated, uncoated or chewable tablet forms. Chewable tablets are preferably a multilayer tablet and still more preferably a bilayer tablet.

The stable compositions of the invention further comprises fillers or diluents, binders, disintegrants or superdisintegrants, complexing agents (cyclodextrin), lubricants, antitacking agent, antiadherants, glidants, surfactants, taste-masking agents, coloring agents, sweeteners and if required, film coating composition, various film coating compositions available from Colorcon under the brand name Opadry®.

In a preferred embodiment, the composition of the invention comprises montelukast sodium, microcrystalline cellulose, specific flavoring agents, diluents, binders, disintegrants, sweeteners, surfactants, lubricants, anti-tacking agents and colorants.

The stable pharmaceutical compositions of the invention comprises montelukast sodium or its other pharmaceutically acceptable salts in the range of about 1% to about 10% by weight of the total composition, preferably from about 2% to about 3% by weight of the total composition.

Microcrystalline cellulose has revolutionized tablet manufacturing because of its unique flow, compressibility and high dilution capacity. It exhibits excellent properties as an excipient for solid dosage forms. Microcrystalline cellulose is a well-known tablet diluent, binder and disintegrant. Its chief advantage over other excipients is that it can be directly compressed into self-binding tablets, which disintegrate rapidly when placed into water or placed on tongue. Other advantages include low friability, inherent lubricity; microcrystalline cellulose is commercially available under the trade name EMCOCEL® from Edward Mendell Co., Inc. and as AVICEL® from FMC Corp.USA. Several grades of microcrystalline cellulose that vary in particle size, density and moisture content are available, for example, AVICEL® PH-101, PH-102, PH-103, PH-105, PH-112, PH-113, PH-200, PH-301, and PH-302. Compared to other directly compressible excipients, microcrystalline cellulose (Avicel PH102) is generally considered to exhibit superior flow, compressibility, binding and disintegration properties. Preferably, the compositions of the present invention comprise microcrystalline cellulose commercially available under the brand name AVICEL PH- 102 from FMC Corp. USA. The compositions of the present invention comprises from about 10% to about 90% of microcrystalline cellulose, preferably from about 20% to about 70% and more preferably, from about 40% to 60% by weight of the composition.

The flavoring agents in accordance with the invention are those flavoring agents, which stabilize the compositions of the present invention by negating the destabilizing impact of microcrystalline cellulose. The flavoring agents stabilizing the composition of the present invention are strawberry, cherry, banana, vanilla and other flavors or mixture thereof. The preferred flavoring agent for the composition of the present invention is strawberry flavor. The compositions of the present invention can comprise from about 0.01% to about 10%, preferably from about 0.1% to about 5%, and more preferably from about 0.1% to about 3% by weight of the total composition.

Unlimited examples of diluents are crystalline cellulose, lactose monohydrate, lactose anhydrous, mannitol, sucrose, fructose, dextrose, calcium carbonate, calcium phosphate, dibasic, tribasic calcium phosphate, calcium sulfate, maltitol, lactitol, confectioner's sugar, compressible sugar, modified starches, and maltodextrins. Preferably diluent is MCC or mannitol or combination thereof. Diluents are preferably used in the range of about 10% to about 90%, preferably from about 20% to about 70%, still more preferably from about 40% to about 60%, by weight of total composition.

Unlimited examples of binders, for use in the present invention, are polyvinylpyrrolidone, copovidone, gelatin, guar gum, polyethylene oxide, pregelatinized starch, (corn, wheat, potato, tapioca) starches, ethyl cellulose, acacia, glucose, microcrystalline cellulose, dextrin, sodium carboxy methylcellulose, mannitol, sucrose, dextrose, icing sugar with starch, methylcellulose, hydroxypropylmethyl cellulose, hydroxypropyl cellulose and low substituted hydroxypropyl cellulose. Binders are preferably used in the range from about 1% to about 90%, preferably from about 1% to about 70% and more preferably, from about 1% to about 60% by weight of the total composition. Microcrystalline cellulose alone or in combination with polyvinyl pyrrolidone (POVIDONE K30) is the preferred choice of binder for the composition of the present invention.

Unlimited examples of disintegrants, including superdisintegrants are alginic acid, microcrystalline cellulose, croscarmellose sodium, crospovidone, low substituted hydroxypropyl cellulose, polacrilin potassium, sodium starch glycolate, modified pregelatinized starch, crospovidone, pullulan, (corn, wheat, potato, tapioca) starch, and sodium carboxymethyl cellulose. Disintegrants are preferably used in the range from about 1% to about 20%, preferably from about 1% to about 15%, still more preferably from about 1% to about 7% by weight of the total composition. Preferred disintegrant for the composition of the present invention is croscarmellose sodium

Optional taste-masking agents for the composition of the invention are inorganic agents, such as, calcium carbonate, magnesium oxide, calcium gluconate, magnesium aluminum silicate, sodium chloride, and magnesium carbonate; organic acids such as citric acids, fumaric acids, stearic acid or its calcium or magnesium salts; membrane polymers such as, amino methacrylate copolymer, ammonio methacrylate copolymer, cellulose acetate, ethyl acetate and methyl methacrylate copolymer dispersion, ethyl cellulose; sequestering agents such as. Beta cyclodextrin, hydroxy propyl betadex, pullulan and methacrylate polymers or copolymers available under the brand name Eudragit®, e.g., Eudragit EPO from ROHM pharma, USA, and Degussa, Germany. Taste-masking agents as mentioned above can be used either alone or in combination. Taste-masking agents are preferably used in the range of about 0% to about 20% by weight of the total composition.

Suitable examples of sweeteners are acesulfame potassium, aspartame, saccharin, sodium and calcium salts of saccharin, dextrates, dextrose, fructose, mannitol, sucrose, sugar syrup, sucralose, compressible sugar, confectioner's sugar, sorbitol, erythritol, maltitol, maltose, and galactose. Aceulfame potassium is the preferred sweetener for the composition of present invention. Sweeteners are used in the range of from about 0.1% to about 10%, preferably from about 0.1% to about 7%, and more preferably from about 0.1% to about 5% by weight of the total composition.

Suitable examples of colorants include but are not limited to ferric oxides such as ferric oxide red, ferric oxide yellow, caramel and other FDC approved colors known to skilled artisan. Coloring agents are used in the range of from about 0.01% to about 5%, preferably from about 0.1% to about 5%, and more preferably from about 0.1% to about 3% by weight of the total composition.

Unlimited examples of lubricants include stearic acid, magnesium and calcium stearate, zinc stearate, hydrogenated and partially hydrogenated vegetable oils, hydrogenated (type I), polyethylene glycol, talc, light mineral oils, polyoxyl 40 stearate, sorbitan trioleate, starch or mixtures thereof. The preferred lubricant for the composition of the present invention is magnesium stearate. Lubricants are incorporated in the range of from about 0.1% to about 10%, preferably from about 0.1% to about 7% and more preferably, from about 0.1% to about 5% by weight of the total composition.

Unlimited examples of surfactants are polysorbate 80, polysorbate 60, polysorbate 20, sodium lauryl sulphate and cetomacrogol. Sodium lauryl sulphate is the preferred choice of surfactant. Preferably, surfactants are used in the range of about 0.01% to about 5% by weight of the total composition.

Suitable examples of antitacking agents, glidants and antiadherants are calcium silicate, magnesium silicate, hydrophobic colloidal silica, colloidal silicon dioxide, and talc. Preferred choice is colloidal silicon dioxide. Antitacking agents or glidants or antiadherants are incorporated in the range of from about 0.1% to about 10%, preferably from about 0.1% to about 7% and more preferably, from about 0.1% to about 3% by weight of the total composition.

If desired, composition of the invention may be film coated with various ready to use film coating compositions available from Colorcon under the brand name Opadry®. A typical film coating composition comprises polyethylene glycol, hypromellose, iron oxide red, and titanium dioxide, available under the brand name Opadry® pink from colorcon, USA. Film coating improves the elegance of the tablet.

### Commercial Sources of Preferred Active and Inert Pharmaceutical Excipients are Listed in the Table Below

| Material | Brand Name | Source |
|---|---|---|
| Montelukast sodium | — | EX.161, EX.146 of |
| | | U.S. Pat. No. 5,565,473 |
| Microcrystalline | Avicel ® PH102 | FMC BioPolymer, |
| cellulose | | Philadelphia, USA |
| Polyvinylpyrrolidone | Kollidon ® K30 | BASF Pharma, Germany |
| Strawberry flavor | — | Firmenich, Geneva, |
| | | Switzerland |
| Mannitol | Pearlitol ® SD | Rouquette Pharma, France |
| | 200 | |
| Silicon dioxide | Aerosil ® 200 | Evonik Degussa, Frankfurt, |
| | | Germany |
| Croscarmellose | Ac-di-sol ® | FMC BioPolymer, |
| sodium | | Philadelphia, USA |
| Acesulfame potassium | Sunette ® | Nutrinova, Germany |
| Red iron oxide | Spectracol ® | Sensient Colors, UK |
| Magnesium stearate | HyQual ® | Mallinckrodt Chemicals, USA |
| Sodium lauryl sulphate | Texapon ® K | Cognis Corporation, Germany |
| Film coating | Opadry ® white | Colorcon, USA |
| composition | or Opadry ® pink | |

In a preferred embodiment the composition of the invention comprises montelukast sodium, as therapeutically active ingredient, microcrystalline cellulose, strawberry flavor, mannitol, croscarmellose sodium, acesulfame potassium, magnesium stearate, silicon dioxide, red iron oxide as colorant. Preferably, the composition is formulated into a solid dosage form. Solid dosage form is preferably a tablet, which is single layer, or multiple layer tablets, preferably a bilayer tablet.

In one preferred embodiment, the composition of invention is a stabilized chewable single or bilayer tablet. The composition of the invention is preferably formulated into solid dosage form by direct compression process. The direct compression process is preferred because of it being economical as it involves less processing steps, reduced labour and power cost. The direct compression process is also useful for drugs which are sensitive to heat and moisture inherent in wet granulation process. Direct compression also avoids high compaction pressures and therefore useful for drugs which are prone to change in its polymorphic forms on application of high compression pressure. Direct compression also facilitate in optimizing the disintegration as each primary drug particle is liberated from tablet mass and is available for dissolution. There is less likelihood of changes in dissolution profiles of tablets manufactured by direct compression process as compared with wet granulation.

In another embodiment, the composition of the present invention can be formulated as orally disintegrating tablet by incorporating mouth feel improving agents such as a combination of microcrystalline cellulose with guar gum, commercially available from FMC corporation, USA under the brand name Avicel® CE15.

In order to demonstrate the superior stability of the composition of the present invention, as compared with composition of the closest prior art, composition of the invention is subjected to accelerated stability testing at 40° C. and about 75% relative humidity for 3 months as per ICH Guidelines. The results in terms of formation of degradation product, montelukast sulfoxide established the unexpected results of the composition of present invention.

In order to validate the stabilizing impact of flavoring agents, active agent (montelukast sodium) excipients (MCC with or without flavoring agents like strawberry, cherry, banana, vanilla, etc) interaction studies are carried out. Results of the studies confirm the stabilizing effect of specific flavoring agents, which include strawberry, cherry, banana, and the like.

Without being bound by any theory, specific flavoring agents which include strawberry, cherry, banana and other flavors or mixtures thereof, of the composition of invention interfere with the destabilizing effect of the microcrystalline cellulose (MCC).

Compositions of the present invention can be prepared, dry granulation (slugging or roller compaction), wet granulation, and direct-compression process. Preferred process is direct compression.

The dry granulation process consists of blending; slugging the ingredients, dry screening, lubrication, and compression. The wet granulation method is used to convert a powder mixture into granules having suitable flow and cohesive properties for tableting. The procedure consists of mixing the powders in a suitable blender followed by adding the granulating solution under shear to the mixed powders to obtain a granulation. The damp mass is then screened through a suitable screen and dried by tray drying or fluidized bed drying. Alternately, the wet mass may be dried and passed through a mill. The overall process includes: weighing, dry powder blending, wet granulating, drying, milling, blending, lubrication, compression and film coating the compressed tablet.

Above-mentioned process can be carried in conventional equipments such as low (octagonal or twin shell blender) and high shear blenders, rapid mixer granulator (RMG), fluid bed processor, and rotating pans.

The process of direct compression simply involves weighing, blending and compression.

Process of manufacturing single layer chewable tablets by direct compression comprises:
1. Sifting montelukast sodium, microcrystalline cellulose, flavoring agent, diluent, disintegrant, binder, coloring agent, lubricant and glidants through a suitable size mesh.
2. blending the flavoring agent with microcrystalline cellulose in a suitable blender and subsequently mixing the resultant blend with montelukast sodium, diluent, disintegrant, binder, and coloring agent.
3. lubricating the blend obtained in step (2) by mixing with lubricant and glidants.
4. compressing the blend obtained in step (3) on a suitable tablet press.

In accordance with preferred embodiment of the present invention there is provided direct compression process for preparing stabilized pharmaceutical composition of montelukast sodium comprises the steps of:
1. Sifting montelukast sodium, microcrystalline cellulose, strawberry flavor, mannitol, croscarmellose sodium, acesulfame potassium, red iron oxide, silicon dioxide and magnesium stearate through a suitable size mesh.
2. Blending strawberry flavor with microcrystalline cellulose in a suitable blender, and subsequently mixing the resultant blend with montelukast sodium, mannitol, croscarmellose sodium, acesulfame potassium, red iron oxide and silicon dioxide,
3. Lubricating the blend obtained in step 2 by mixing with magnesium stearate, and
4. Compressing the blend obtained in step 3 on a suitable tablet press.

In accordance with the present invention, the stable chewable bi layer tablet comprising an active layer and an inactive layer, wherein:
(i) active layer comprises montelukast sodium and other pharmaceutically acceptable excipients.
(ii) inactive layer comprises other pharmaceutically acceptable excipients; wherein the microcrystalline is incorporated in either of the layer.

Further, process of manufacturing bilayer chewable tablets by direct compression comprises:
(a) Process of manufacturing active layer, which comprises following steps:
   1. Sifting montelukast sodium, diluent, disintegrant, coloring agent, flavoring agent, lubricant and glidant etc through a suitable size mesh.
   2. blending montelukast sodium with flavoring agent in a suitable blender.
   3. mixing the blend of step-2 with diluent, disintegrant, coloring agent.
   4. lubricating the blend of step-3 with lubricant and glidant.
(b) Process of manufacturing inactive layer, which comprises following steps:
   1. Sifting, microcrystalline cellulose, disintegrant, flavoring agent, sweetening agent lubricant and glidant etc through a suitable size mesh.
   2. blending microcrystalline cellulose with flavoring agent in a suitable blender and subsequently mixing the resultant blend with disintegrant and sweetening agent.
   3. lubricating the blend of step-2 with lubricant and glidant.

Finally, compressing the blend obtained in step (a)(4) and step (b)(3) on a suitable bilayer-tableting machine produces the bilayer chewable tablet.

The compression force can be selected based on the type/model of press, what physical properties are desired for the tablets product (e.g., desired, hardness, friability, in vivo disintegration or dissolution characteristics, etc.), the desired tablet appearance and size. In a preferred embodiment, the tablets from the compression stage typically have a hardness of about 5 Kp to about 25 Kp.

The dissolution rate of the composition of the present invention of about at least 80% by weight in about 45 minutes in accordance with the USP paddle dissolution test <711> on pages 1791-1793 of USP 23/nf 18,1995, United states pharma-copeial convention, Inc Rockville Md. 20852. Dissolution rate is measured in 900 ml of water having 0.5% sodium lauryl sulphate at 37° C. The preferred dissolution rate is at least about 80% by weight of montelukast sodium released in about 30 minutes, and still more preferably, the dissolution rate of the composition of present invention is at least about 90% by weight of montelukast sodium released in about 30 minutes. Dissolution rate of the composition of the present invention is comparable with the corresponding marketed formulation of montelukast in USA, Singulair®.

The chewable tablets of the present invention thus produced typically exhibit sufficient hardness and low friability and are adapted for packaging in HDPE bottles or push-through blister packs. The tablets are robust enough to avoid breakage during storing, distribution and transportation.

The following examples further describe and demonstrate embodiments within the scope of the present invention. These examples are given solely for the purpose of illustration and are not to be construed as a limitation of the present invention, as many variations thereof are possible without departing from the spirit and scope.

### EXAMPLE 1

### Drug-Excipient Interaction Studies

In order to validate the stabilizing effect of specific flavoring agents, active (montelukast sodium) excipients interaction studies were carried out in glass vials. Each composition was stored at about 55° C. for 48hrs.

The percentage by weight of montelukast sulfoxide degradation product was expressed in terms of labeled (initial) amount of montelukast. Montelukast sulfoxide degradation product was analysed in each sample by validated HPLC method. HPLC was performed on a column made up of Zorbax Eclipse XBD, C-18 (150*4.6) mm, packed with 5 μm diameter particles. Mobile phase was (a) sodium dihydrogen orthophosphate at pH 3 (b) Acetonitrile; flow rate was 1.0 ml/min. The UV detector was set at 225 nm and the column temperature was 30° C. The results are presented in table below.

TABLE 1
Stability data of montelukast sodium with different excipients

| | | % of montelukast | |
|---|---|---|---|
| | | sulfoxide degradation | |
| | | product | |
| | | | when kept at |
| S.No. | Composition | Initial | 55° C. for 48 hrs |
| 1. | Montelukast sodium | 0.031 | 0.032 |
| 2. | Montelukast sodium + microcrystalline | 0.04 | 3.18 |
| | cellulose (1:10) | | |
| 3. | Montelukast sodium + microcrystalline | 0.06 | 2.06 |
| | cellulose + cherry (1:10:1) | | |
| 4. | Montelukast sodium + microcrystalline | 0.03 | 2.08 |
| | cellulose + banana (1:10:1) | | |
| 5. | Montelukast sodium + microcrystalline | 0.05 | 2.80 |
| | cellulose + vanilla (1:10:1) | | |
| 6. | Montelukast sodium microcrystalline | 0.06 | 2.05 |
| | cellulose + strawberry (1:10:1) | | |

It is clearly evident from above mentioned data that specific flavoring agents like strawberry, cherry, banana and the like induce significant reduction in the formation of montelukast sulfoxide degradation product.

### EXAMPLE 2

### Chewable Montelukast Tablet

| S.No. | Ingredients | Mg/Tab |
|---|---|---|
| 1. | Montelukast sodium | 5.2 |
| 2. | Microcrystalline cellulose Ph 102 | 131.80 |
| 3. | Mannitol | 132.00 |
| 4. | Silicon dioxide | 1.50 |
| 5. | Croscarmellose sodium | 15.00 |
| 6. | Acesulfame potassium | 3.00 |
| 7. | Red Iron oxide | 1.50 |
| 8. | Strawberry flavor | 1.00 |
| 9. | Magnesium stearate | 9.00 |
| | Total | 300.00 |

### Manufacturing Procedure:

1. Montelukast sodium, microcrystalline cellulose, mannitol, silicon dioxide, croscarmellose sodium, acesulfame potassium, red iron oxide, strawberry flavor and magnesium stearate were accurately weighed and sifted through ASTM sieve no. 60.
2. Strawberry flavor was blended with microcrystalline cellulose in conta blender for 10 min.
3. Red iron oxide was first blended with silicon dioxide and then blended in geometric ratio with montelukast sodium, acesulfame potassium, mannitol and croscarmellose sodium in conta blender for 10 min.
4. Blend obtained in step 2 was mixed with blend obtained in step 3 in low shear mixer.
5. Resultant blend of step 4 was lubricated with magnesium stearate for 3 min.
6. Lubricated blend obtained in step-5 was finally compressed using suitable tableting machine.

### EXAMPLE 3

### Chewable Montelukast Tablet

| S.No. | Ingredients | Mg/Tab |
|---|---|---|
| 1. | Montelukast sodium | 5.2 |
| 2. | Microcrystalline cellulose PH102 | 132.30 |
| 3. | Mannitol | 130.00 |
| 4. | Silicon dioxide | 1.50 |
| 5. | Croscarmellose sodium | 15.00 |
| 6. | Acesulfame potassium | 3.00 |
| 7. | Red Iron oxide | 1.50 |
| 8. | Sodium Lauryl sulphate | 1.50 |
| 9. | Strawberry flavor | 0.50 |
| 10. | Peppermint flavor | 0.50 |
| 11. | Magnesium stearate | 9.00 |
| | Total | 300.00 |

### Manufacturing Procedure:

1. Montelukast sodium, microcrystalline cellulose, mannitol, silicon dioxide, croscarmellose sodium, acesulfame potassium, red iron oxide, sodium lauryl sulphate, strawberry flavor, peppermint flavor and magnesium stearate were accurately weighed and sifted through ASTM sieve no. 60.
2. Strawberry flavor and peppermint flavor were blended with microcrystalline cellulose in conta blender for 10 min.
3. Red iron oxide was first blended with silicon dioxide and then blended in geometric ratio with montelukast sodium, acesulfame potassium, mannitol, sodium lauryl sulphate and croscarmellose sodium in conta blender for 10 min.
4. Blend obtained in step 2 was mixed with blend obtained in step 3 in low shear mixer.
5. Resultant blend of step 4 was lubricated with magnesium stearate for 5 min.
6. Lubricated blend obtained in step 5 was finally compressed using suitable tableting machine.

### EXAMPLE 4

### Chewable Montelukast Tablet

| S.No. | Ingredients | Mg/Tab |
|---|---|---|
| 1. | Montelukast sodium | 5.27 |
| 2. | Microcrystalline cellulose Ph 102 | 180.00 |
| 3. | Mannitol | 83.73 |
| 4. | Silicon dioxide | 1.50 |
| 5. | Croscarmellose sodium | 15.00 |
| 6. | Acesulfame potassium | 3.00 |
| 7. | Red Iron oxide | 1.50 |
| 8. | Strawberry flavor | 1.00 |
| 9. | Magnesium stearate | 9.00 |
| | Total | 300.00 |

### Manufacturing Procedure

1. Montelukast sodium, microcrystalline cellulose, mannitol, silicon dioxide, croscarmellose sodium, acesulfame potassium, red iron oxide, strawberry flavor and magnesium stearate were accurately weighed and sifted through ASTM sieve no. 60.
2. Strawberry flavor was blended with microcrystalline cellulose in conta blender for 10 min.
3. Red iron oxide was first blended with silicon dioxide and then blended in geometric ratio with montelukast sodium, acesulfame potassium, mannitol, sodium lauryl sulphate and croscarmellose sodium in conta blender for 10 min.
4. Blend obtained in step 2 was mixed with blend obtained in step 3 in low shear mixer.
5. Resultant blend of step 4 was lubricated with magnesium stearate for 5 min.
6. Lubricated blend obtained in step-5 was finally compressed using suitable tableting machine.

### EXAMPLE 5

### Chewable Montelukast Tablet

| | | |
|---|---|---|
| S.No. | Ingredients | Mg/Tab |

Compaction composition

| | | |
|---|---|---|
| 1. | Montelukast sodium | 5.2 |
| 2. | Mannitol | 150.00 |
| 3. | Silicon dioxide | 1.00 |
| 4. | Croscarmellose sodium | 7.50 |
| 5. | Red Iron oxide | 1.00 |
| 6. | Aspartame | 3.00 |
| 7. | Sodium lauryl sulphate | 3.00 |
| 8. | Polyvinylpyrollidone (kollidon 30) | 3.00 |

Extragranular composition

| | | |
|---|---|---|
| 10. | Microcrystalline cellulose Ph 102 | 107.72 |
| 11. | Croscarmellose sodium | 7.5 |
| 12. | Silicon dioxide | 0.5 |
| 13. | Red Iron oxide | 0.5 |
| 14. | Peppermint flavor | 1.0 |
| 15. | Magnesium stearate | 9.0 |
| | Total | 300.00 |

### Manufacturing Procedure

1. Montelukast sodium, mannitol, silicon dioxide, croscarmellose sodium, red iron oxide, aspartame, sodium lauryl sulphate, and polyvinylpyrollidone were accurately weighed and sifted through suitable size mesh.
2. Ingredients of step-1 were blended in geometric proportion.
3. Blend of step-2 was compacted in a roller compacter.
4. Compact formed in step-3 was passed through a suitable size mesh to form compacted granules.
5. Microcrystalline cellulose, croscarmellose sodium, silicon dioxide, red iron oxide, peppermint flavor and magnesium stearate were passed through a suitable size mesh and blended in a geometric ratio.
6. Peppermint flavor was blended with microcrystalline cellulose in a suitable blender
7. Croscarmellose sodium, silicon dioxide, red iron oxide were blended in geometric ratio.
8. Blend of step 7 was mixed with blend obtained in step 6.
9. Resultant blend of step 8 was mixed with granules obtained in step 4.
10. Blend obtained in step-9 was lubricated with magnesium stearate and was finally compressed using suitable tableting machine.

### EXAMPLE 6

### Chewable Montelukast Bilayer Tablet

| | | |
|---|---|---|
| S.No. | Ingredients | Mg/Tab |

Active layer

| | | |
|---|---|---|
| 1. | Montelukast sodium | 5.27 |
| 2. | Mannitol | 133.98 |
| 3. | Red Iron oxide | 0.75 |
| 4. | Croscarmellose sodium | 7.00 |
| 5. | Magnesium stearate | 3.00 |
| | Total | 150.00 |

Inactive layer

| | | |
|---|---|---|
| 6. | Microcrystalline cellulose Ph 102 | 135.00 |
| 7. | Acesulfame potassium | 3.00 |
| 11. | Croscarmellose sodium | 8.00 |
| 12. | Strawberry flavor | 1.00 |
| 13. | Magnesium stearate | 3.00 |
| | Total | 150.00 |

### (a) Manufacturing Procedure of Active Layer:

1. Montelukast sodium, mannitol, red iron oxide, croscarmellose sodium and magnesium stearate were accurately weighed and passed through a suitable size mesh.
2. The ingredients of step-1 were blended in a geometric ratio in a suitable blender.

### (b) Manufacturing Procedure of Inactive Layer

1. Strawberry flavor was blended with microcrystalline cellulose in a suitable blender.
2. The blend obtained in step 1 was mixed with acesulfame potassium and croscarmellose sodium.
3. The resultant blend of step 2 was lubricated with magnesium stearate.

The blends obtained in step (a)2 and steps (b)3 were finally compressed to make a bilayer tablet.

### EXAMPLE 7

### Chewable Montelukast Tablet

| S.No. | Ingredients | Mg/Tab |
|---|---|---|
| 1. | Montelukast sodium | 5.27 |
| 2. | Hydroxypropyl cellulose LH-11 | 6.81 |
| 3. | Sodium starch glycolate | 13.62 |
| 4. | Aspartame | 1.35 |
| 5. | Mannitol | 262.12 |
| 6. | Red Iron oxide | 1.35 |
| 7. | Cherry flavor | 5.4 |
| 8. | Magnesium stearate | 4.08 |
| | Total | 300.00 |

### Manufacturing Procedure

1. Montelukast sodium, hydroxypropyl cellulose LH-11, sodium starch glycolate, aspartame, mannitol, red iron oxide, cherry flavor and magnesium stearate were accurately weighed and sifted through a suitable size mesh.
2. red iron oxide was blended with mannitol in a geometric ratio in a suitable blender.
3. montelukast sodium was blended with material obtained in step 2.
4. blend obtained in step-3 was mixed with other ingredients of step-1.
5. blend obtained in step-4 was lubricated with magnesium stearate and finally compressed into tablet.

### EXAMPLE 8

Montelukast sodium chewable tablets of example 2, montelukast sodium chewable tablets prepared as per example 6 of US20070184108 and Chewable Singulair® tablet were placed in glass vials and stored at 55° C. for 48 hrs. Samples were analyzed by validated HPLC method. HPLC was performed on a column made up of Zorbax Eclipse XBD, C-18 (150*4.6) mm, packed with 5 μm diameter particles. Mobile phase was (a) sodium dihydrogen orthophosphate at pH 3 (b) Acetonitrile; flow rate was 1.0 ml/min. The UV detector was set at 225 nm and the column temperature was 30° C.

The amount of montelukast sulfoxide degradation product formed is expressed as the percentage by weight of labeled (initial) amount of montelukast in the composition. The results are set out in table 2.

TABLE 2
Stability data of Montelukast sodium compositions at 55° C. for 48 hrs

| | % of montelukast sulfoxide | |
|---|---|---|
| | degradation product | |
| | | When stored at |
| Formulation | Initial | 55° C. for 48 hrs |
| Chewable Singulair ® tablet | 0.49 | 0.88 |
| Example 6 of US20070184108 | 0.20 | 2.05 |
| Example 2 (with MCC + | 0.035 | 0.287 |
| strawberry) of present invention | | |

### EXAMPLE 9

Montelukast sodium chewable tablets of example 2 were placed in high-density polyethylene (HDPE) bottles or blister packages and subjected to accelerated storage condition of 40° C. temperature and 75% relative humidity for 3 months. Samples were analyzed by validated HPLC method. HPLC was performed on a column made up of Zorbax Eclipse XBD, C-18 (150*4.6) mm, packed with 5 μm diameter particles. Mobile phase was (a) sodium dihydrogen orthophosphate at pH 3 (b) Acetonitrile; flow rate was 1.0 ml/min. The UV detector was set at 225 nm and the column temperature was 30° C.

The amount of montelukast sulfoxide degradation product formed is expressed as the percentage by weight of labeled (initial) amount of montelukast in the composition The results obtained were compared with the results given in table 2 for example-4, of application number, US20070184108 and commercially available (in USA) chewable montelukast tablet, Singulair®, from Merck & Co. The results are set out in table 3.

TABLE 3
Stability data of Montelukast sodium compositions at 40° C./75% RH

| | % of montelukast sulfoxide | |
|---|---|---|
| | degradation product | |
| | | After 3 months when |
| | | stored at 40° C./75% |
| Formulation | Initial | RH |
| Chewable Singulair ® | 0.49 | 1.14 |
| tablet | | |
| Example 4 of | 0.1 | 0.2 |
| US20070184108 | | |
| Example 2 of | 0.035 | 0.035 |
| present invention | | |

The above-mentioned data establish that pharmaceutical compositions of the present invention are stabilized in terms of formation of montelukast sulfoxide degradation product. It is validated that degradation impurity guideline of International Conference on Harmonization for a 10 mg tablet is also complied.

### EXAMPLE 10

To determine the release of montelukast sodium from the pharmaceutical composition of the present invention, the chewable tablets of Example 2 were subjected to dissolution study. The dissolution profile from chewable tablets of Example 2 was compared with the dissolution profile of commercially available chewable Singulair® tablets by Merck & Co., Inc. The results are presented in table 4 as a mean percentage release of the total montelukast sodium contents from the chewable tablets. Dissolution study parameters were as follows:
Apparatus: USP type II; 50 RPM
Time: 30 minutes
Medium: 0.5 SLS in water, 900 ml, 37° C.±2° C.

TABLE 4
Dissolution profile

| | Mean percentage release of montelukast sodium | |
|---|---|---|
| Time (min) | Chewable Singulair ® | Chewable tablets of Example 2 |
| 5 | 100 | 86 |
| 10 | 102 | 90 |
| 20 | 104 | 92 |
| 30 | 104 | 94 |

From the above tabular data, it is clearly evident that the dissolution profile of chewable tablets of the invention (Example.2) is comparable to the dissolution profile of commercially available (in USA) chewable Singulair® tablets by Merck & Co., Inc. It also complies with USP guidelines for chewable tablets i.e. ≧ or 80% release of the labeled (initial) amount of montelukast in 30 minutes.

Many modifications and variations of this invention can be made without departing from scope of this invention, as will be apparent to those skilled in the art. Invention is to be limited only by the terms of the appended claims, along with the full scope of the equivalents to which claims are entitled.

1. A stable pharmaceutical composition comprising a therapeutically effective amount of montelukast or its pharmaceutically acceptable salts, solvates or hydrates and a blend comprising microcrystalline cellulose and a flavoring agent, wherein said composition contains less than about 0.1% by weight of montelukast sulfoxide degradation product after storage at about 40° C. and about 75% relative humidity for three months.

2. The stable pharmaceutical composition of claim 1, wherein the flavoring agent is selected from a group consisting of strawberry, cherry, banana, vanilla and peppermint or mixture thereof.

3. A stable pharmaceutical composition comprising a therapeutically effective amount of montelukast or its pharmaceutically acceptable salts, solvates or hydrates and a blend comprising microcrystalline cellulose and a strawberry flavor, wherein said composition contains less than about 0.1% by weight of montelukast sulfoxide degradation product after storage at about 40° C. and about 75% relative humidity for three months.

4. The stable pharmaceutical composition of claim 3, wherein the composition contains less than about 0.04% by weight of montelukast sulfoxide degradation product after storage at about 40° C. and about 75% relative humidity for three months.

5. A stable pharmaceutical composition comprising a therapeutically effective amount of montelukast or its pharmaceutically acceptable salts, solvates or hydrates and a blend comprising microcrystalline cellulose and a flavoring agent, wherein the composition contains less than about 1% by weight of montelukast sulfoxide degradation product after storage at about 55° C. for 48 hours.

6. The stable pharmaceutical composition of claim 5, wherein the flavoring agent is selected from strawberry, cherry, banana, vanilla and peppermint or mixture thereof.

7. The stable pharmaceutical composition of claim 5, wherein the composition contains less than about 0.3% by weight of montelukast sulfoxide degradation product after storage at about 55° C. for 48 hours.

8. The stable pharmaceutical composition of claim 1, wherein the montelukast is used in the form of montelukast sodium.

9. The stable pharmaceutical composition of claim 1, wherein the composition further comprising pharmaceutical acceptable excipients selected from diluents, binders, disintegrants, sweeteners, surfactants, lubricants, anti-tacking agents or colorants or mixture thereof.

10. The stable pharmaceutical composition of claim 9, wherein the composition further comprising mannitol, croscarmellose sodium, acesulfame potassium, red iron oxide, magnesium stearate or silicon dioxide or mixture thereof.

11. The stable pharmaceutical composition of claim 1, wherein the composition is in the form of a tablet.

12. The stable pharmaceutical composition of claim 11, wherein the tablet is a chewable tablet.

13. The stable pharmaceutical composition of claim 12, wherein the chewable tablet is a bilayer tablet.

14. The stable pharmaceutical composition of claim 11, wherein the tablet is prepared by a process comprising dry granulation, wet granulation or direct compression.

15. The stable pharmaceutical composition of claim 12, wherein the tablet is prepared by a process comprising:
a. sifting montelukast salt, solvates or hydrates thereof, microcrystalline cellulose, flavoring agent, diluent, disintegrant, binder, coloring agent, lubricant and glidants through a suitable size mesh;
b. blending flavoring agent with microcrystalline cellulose in a blender and subsequently mixing the resultant blend with montelukast sodium, diluent, disintegrant, binder, and coloring agent;
c. lubricating the resultant blend by mixing with lubricant and glidants; and
d. compressing the resultant blend obtained on a suitable tablet press.

16. The stable pharmaceutical composition of claim 1, wherein the composition is prepared by a process comprising:
a. providing montelukast salt, solvates or hydrates;
b. blending the flavoring agent with microcrystalline cellulose in a blender and subsequently mixing the resultant blend with montelukast sodium, solvates or hydrates; and
c. preparing the composition enabling the stable pharmaceutical composition.

17. The stable pharmaceutical composition of claim 3, wherein the composition is prepared by a process comprising:
a. providing montelukast salt, solvates or hydrates;
b. blending strawberry flavor with microcrystalline cellulose in a blender and subsequently mixing the resultant blend with montelukast sodium, solvates or hydrates; and
c. preparing the composition enabling the stable pharmaceutical composition.
